(19) United States
(12) Patent Application Publication
(10) Pub. No.: US 2008/0164337 A1
(43) Pub. Date: Jul. 10, 2008

(54) Volatile Liquid Disseminating Device

(75) Inventors: Colin Brown, Bracknell Berkshire (GB); Guy Edward Naish, Bicester Oxfordshire (GB); Kishen Gohil, New Malden Surrey (GB)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 11/813,577

(22) PCT Filed: Jan. 6, 2006
(86) PCT No.: PCT/CH2006/000011

§ 371 Date: Jul. 9, 2007

(30) Foreign Application Priority Data
Jan. 12, 2005 (GB) 0500481.7

(51) Int. Cl. A61L 9/04

(52) U.S. Cl. 239/34

(57) ABSTRACT

An apparatus adapted to provide in an atmosphere one of at least three volatile liquids, the apparatus comprising a source of air current, radially extending from the source of air current a plurality of coplanar air channels, corresponding to the number of volatile liquids, placed such that the air current flows through them, each channel leading to a different volatile liquid-emitting member and then into the atmosphere; and located in each channel between the source and the volatile liquid-emitting member, means of blocking the channel and preventing the flow of air therethrough. The apparatus allows an easy change from one liquid to another and control over the time of the emission of each liquid.

This invention relates to apparatus for disseminating volatile liquids in vapour form into an atmosphere.

Apparatus for dissemination of a volatile substance, such as a fragrance, an odour masking agent or an insecticide, into an atmosphere are well known and widely used. These apparatus are generally simple and therefore cheap to manufacture, but they are universally for single fragrances. They are inadequate for more sophisticated applications where a number of fragrances are required, especially when it is desirable that fragrances be selectable.

There already exist on the market apparatus that can disseminate a number of fragrances and that permit the selection of which fragrance will be disseminated at which time. They generally involve a fan and a number of containers, the containers having suitable disseminating members (such as porous wicks allowing the transport of the liquid between a storage reservoir and the atmosphere) and being movable with respect to the fan. Thus, when a particular fragrance is desired, the appropriate container and disseminating member is moved into position with respect to the fan, such that the air current from the fan evaporates the volatile liquid and conveys it to the atmosphere.

While such apparatus are generally satisfactory in performance, they have several drawbacks. One is that the mechanism for moving the various containers is complex and therefore expensive and more prone to mechanical failure. Another drawback is that such an apparatus requires the use of fragrances in a particular sequence, the one in which they are mounted. It is possible to utilise fragrances out of sequence, but this involves a further undesirable degree of mechanical and electrical complexity.

Attempts to overcome these and similar problems have resulted in sophisticated apparatus. Examples of such apparatus include Japanese Kokai H03-139630 and Kokai 2003-310740, UK published application GB 2 041 407 and PCT published application WO 2003/028775. All of these are relatively complex and therefore relatively expensive.

It has now been found that a relatively simple, robust apparatus can be used to disseminate a plurality of individual volatile liquids into an atmosphere. The invention therefore provides an apparatus adapted to provide in an atmosphere one of at least three volatile liquids, the apparatus comprising:
(a) a source of air current;
(b) radially extending from the source of air current a plurality of coplanar air channels corresponding to the number of volatile liquids, placed such that the air current flows through them, each channel leading to a different volatile liquid-emitting member and then into the atmosphere; and
(c) located in each channel between the source and the volatile liquid-emitting member, means of blocking the channel and preventing the flow of air therethrough.

The invention further comprises a method of disseminating into an atmosphere at least one volatile liquid selected from at least three such liquids provided in the apparatus, comprising the passing of an air current from a source of air current through a plurality of coplanar air channels corresponding in number to the number of volatile liquids, the individual channels extending radially from the source and leading to a different volatile liquid-emitting member and then to the atmosphere, the channels being able individually to be blocked between the source and the volatile liquid-emitting member.

The source of air current may be any suitable source, typically a fan, vane or an impeller (hereinafter generally referred to as “fan”), rotated by any suitable means, for example, by an electric motor on whose shaft it is mounted. The motor may be powered by any suitable power source, such as battery, mains electricity or solar cells. In one aspect of the invention, the fan is one that causes an air current that is coplanar with the fan, that is, radial from the axis of rotation of the fan.

The plurality of channels is positioned so as to be in the air current. In the embodiment referred to hereinabove in which the air current is coplanar with the fan, the channels are placed radially around the circumference of the fan, preferably equidistant from each other. The number of channels will depend on the number of volatile liquids desired, but it is at least three, as known simpler devices can easily manage two volatile liquids. A typical number is between 3 and 8, and in a further aspect between 4 and 6; this permits a desirable number of volatile liquids along with a desirable combination of reasonable liquid quantity of internal reservoirs and compact dimensions. It is possible of course to have larger reservoirs remote from the apparatus and supplying the apparatus by means of feeder tubes. This increases the number of possible volatile liquids substantially, but it also increases considerably the complexity of the apparatus, and while such an apparatus may be appropriate for large institutions, it is not appropriate for most uses, where a relatively simple, self-contained, easily replenished apparatus is desirable.

Thus, in one particular embodiment, the volatile liquids are held in replaceable reservoirs, allowing for easy replenishment.

In the embodiment in which the air current is coplanar with the fan, the channels form part of a body that surrounds at least a substantial part (or even all) of the circumference of the fan. They may be provided by any suitable means in any suitable material, for example, by casting, machining and moulding a material such as a plastic, a metal or a ceramic.

Each channel leads to a volatile liquid-emitting member and from there into the atmosphere. The volatile liquid-emitting member may be any suitable such member. For example, it may be a spray nozzle. However, for simplicity and cheapness, there is used an evaporative surface, such as a porous wick of any suitable material, with one end in a reservoir of volatile liquid and the other in the air stream. Such wicks are well known to the art and are widely available. Alternatively, the liquid-emitting member may be a flat pad of any suitable material, connected to a reservoir of volatile liquid. The volatile liquid may be transferred to the volatile liquid -emitting member by any convenient means, one such means being by capillary action.

The reservoirs and/or the volatile liquid-emitting members may optionally be equipped with heating elements, which assist in the evaporation of liquid into the air stream.

In each channel, between the source of air current and the volatile liquid-emitting member, is provided a means of blocking the channel. This can take any suitable form, and the skilled person will recognise the many kinds of suitable blocking means that are possible. Without limiting the scope of the invention in any way, one advantageous blocking means is electromagnetic in operation. Typical examples include a solenoid valve and an electromagnet that, when switched on, attracts a ferromagnetic material, causing it to move and thus to block (or unblock) a channel. Reopening of the channel may also be by any convenient means, such as gravity acting on a ferromagnetic element that has been raised against the force of gravity by an electromagnet, the urging of a compressed spring or the actuation of a solenoid valve.

The actuation and control of the blocking means and other aspects of the apparatus, for example, the air current speed, may be by any suitable means, and again the skilled person will see the many possibilities within the skill of the art. For example, it may be achieved by means of a pre-set program in the apparatus itself. This may be completely automatic, or it may be capable of being overridden manually by adjusting the apparatus directly, for example, by means of a suitable control panel provided in the apparatus. Such a system may allow a variety of possibilities, such as fragrance selection, order and time of emission and so on. All of these are well within the skill of the art.

Alternatively, actuation and control may be achieved by remote control. One such means of control is a control cable leading from a control unit to the apparatus. The control unit may be a computer or may have a computer associated with it, and instructions may be input into the computer, either directly or via the Internet. However, the control may be wireless, for example, by electromagnetic signal, such as an infra-red apparatus of the type used for television and other domestic entertainment electronic equipment. However, it may also be radio frequency equipment. Such a control unit may be equipped with any necessary control means, for example, means for opening and closing any blocking means or combination of blocking means, means for adjusting the speed of rotation of the air current and means for adjusting the temperature of any heating element. These can all be achieved by readily-available components. The instructions may come, for example, from a dedicated controller, or they may come to a computer equipped with a radio transmitter, either by direct input or over the Internet, or by means of an SMS message from a mobile telephone.

The apparatus according to this invention may be easily manufactured using common materials and techniques. It is versatile and reliable in use, easy to replenish, and it allows the emission into an atmosphere of a selected volatile liquid or combination of such liquids. Any volatile liquid useful for dissemination into an atmosphere in vapour form may be used. The most significant group of such liquids are fragrances, and the apparatus according to this invention can be used to change the fragrance, and therefore the atmosphere or “mood” of a room. Another possible use is use with an audio-visual display, in which a fragrance can be released to match what is on the screen. However, other liquids can also be used, for example, disinfectants, insecticides and fungicides. For example, an apparatus can contain both an insecticide and a fragrance, such that the insecticide release can be remotely triggered, and when that has had sufficient time to work, a fragrance can be released to cover the odour of the insecticide.

The invention is further described with reference to any one of the drawings which depict preferred embodiments and which are not intended to be limiting in any way. The skilled person will be able to envisage many other ways of carrying the invention into effect, all of which lie within the scope of the invention.

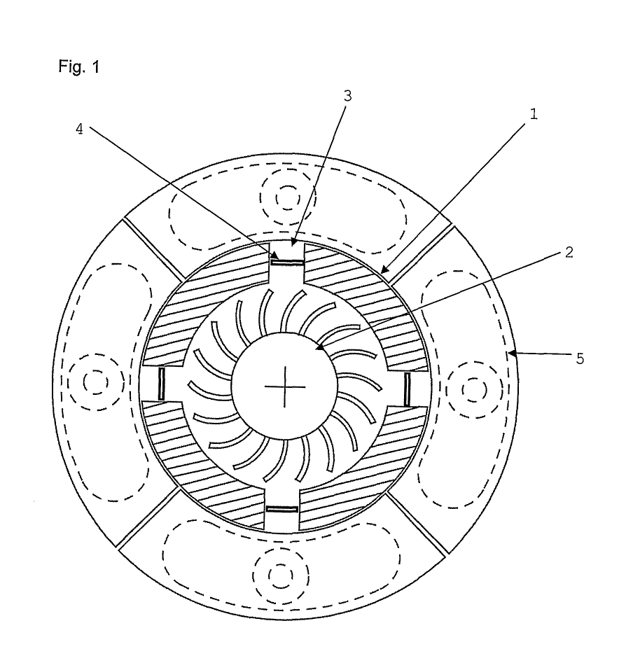

FIG. 1 depicts a transverse cross-section through an apparatus.

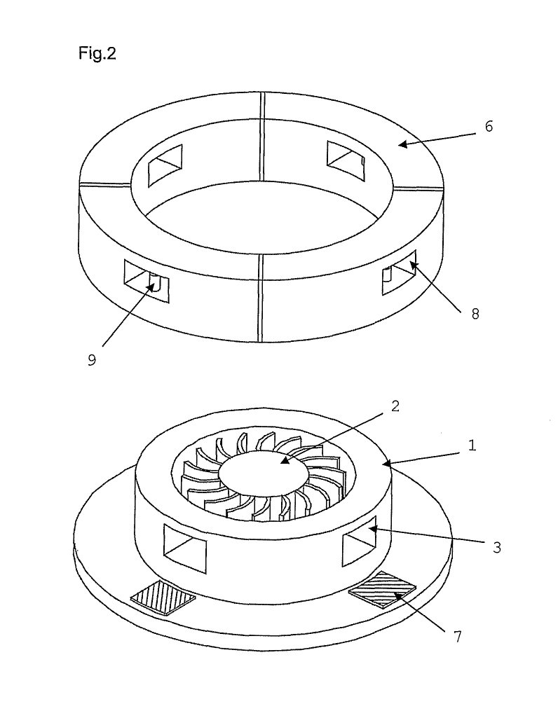

FIG. 2 depicts a perspective exploded view of the embodiment of FIG. 1.

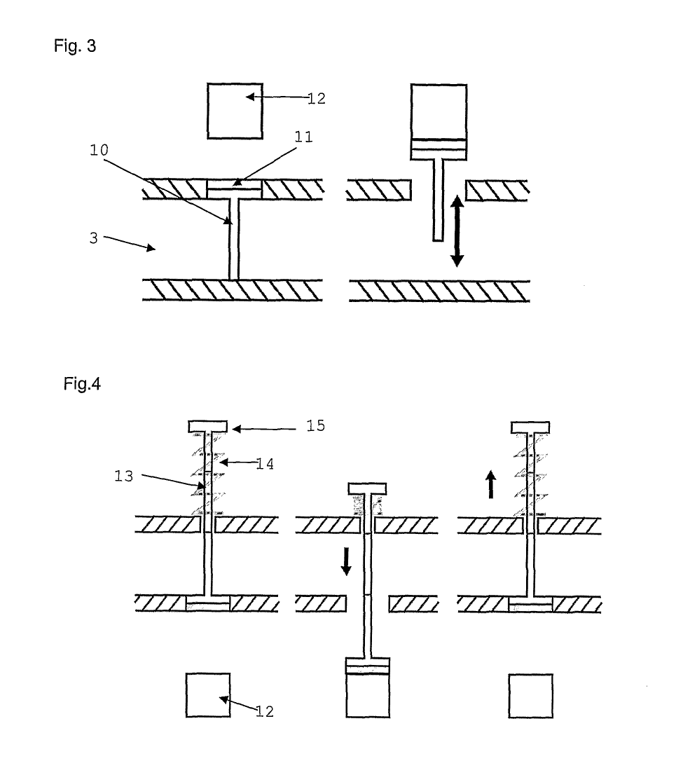

FIG. 3 is a schematic representation of one form of blocking means.

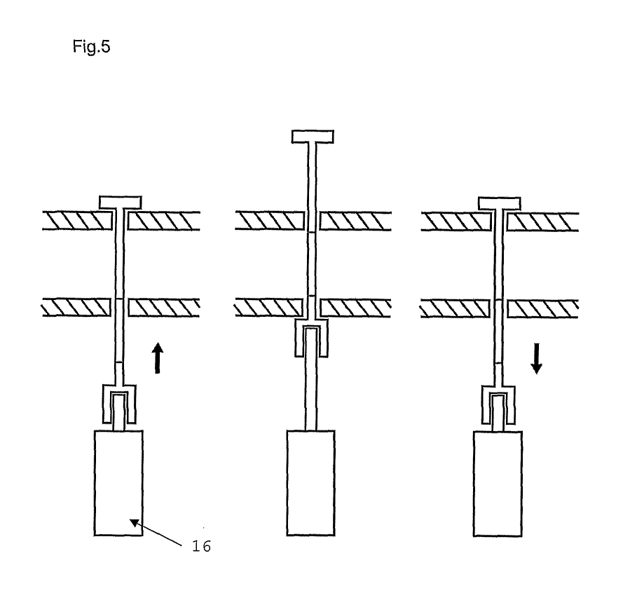

FIG. 4 is a schematic representation of another form of blocking means.

In FIGS. 1 and 2, an essentially toroidal member 1 surrounds a centrifugal fan 2 and lies in the plane of rotation of the fan. In the toroidal member are formed four channels 3, arranged equidistantly around the toroidal member and extending radially from the fan. Within each channel 3 is a blocking means 4. Surrounding the toroidal member is a further toroidal member 6, which has four channels 8 that form continuations of the four channels 3. Within the further toroidal member 6 are four reservoirs of fragrance (shown in ghosted outline at 5) with associated fragrance-emitting means 9, in this case porous wicks leading from the reservoir and protruding into the channel 8. The toroidal member 1 also has mounted thereon heating elements 7, one for each reservoir, such that, when the apparatus is assembled, each reservoir 5 sits on a heating element. If desired, the heating element can be switched on and assist in the transport and evaporation of the fragrance.

In operation, the fan 2 blows air radially outwards. The air is blown through the channels 3, and, if the blocking means 4 for the particular channel is open, past the wick 9, where it evaporates and entrains fragrance, and then through channel 8 into the atmosphere.

In FIG. 3, the blocking means has the form of a gate valve 10, which blocks the channel 3 and which is able to be lifted vertically to such an extent that it clears the channel 3. At the top of the valve is a piece of ferromagnetic material (typically steel) 11, which is in close proximity to an electromagnet 12. When the electromagnet 12 is energised, the ferromagnetic material is attracted to it, and the gate valve is thus raised and the channel 3 opened. When the electromagnet is no longer energised, gravity causes the gate valve to close.

FIG. 4 depicts a variant of the FIG. 3 valve. In this case, the gate valve is attracted downwards by the electromagnet 12 and it has a port 13 that is brought into alignment with the channel by the downwards movement. It is closed again by means of a helical spring 14 fitted to an elongated stem of the gate valve and which seeks to pull the gate valve upwards and away from the electromagnet. It does this when the electromagnet is no longer energised.

The valve of FIG. 5 is a variation on that of FIG. 4, in this case being operated by a solenoid switch 16 positioned beneath the gate valve. When the switch is activated, it pushes the gate valve upwards, this bringing the valve port into alignment with the channel, thus opening it. When it is deactivated, the switch returns to its rest position and the gate closes.

1. An apparatus adapted to provide in an atmosphere one of at least three volatile liquids, the apparatus comprising:
(a) a source of air current providing an air current flow;
(b) a plurality of coplanar air channels radially extend from the source of air current and correspond to a plurality of reservoirs containing volatile liquids, the channels are arranged such the air current to flows through them a plurality of second channels into the atmosphere, each of the plurality of second channels being connected to one of the reservoirs containing volatile liquid-emitting member; and
(c) blocking means are disposed located in each air channel for selectively preventing the air current flow therethrough.

2. An apparatus according to claim 1, wherein the source of air current is a fan or impeller.

3. The apparatus according to claim 2, wherein the fan or impeller rotates in the plane of the plurality of air channels and the plurality of second channels.

4. The An apparatus according to claim 1, wherein the plurality of air channels and the plurality of second channels are each 3-8, preferably 4-6.

5. The apparatus according to claim 1, wherein the liquid-emitting member comprises an evaporative surface, protruding into the air current flow.

6. The An apparatus according to claim 1, wherein the blocking means are electromagnetically actuated.

7. The apparatus according to claim 6, wherein the blocking means are doors operated by solenoid switches.

8. The apparatus according to claim 1, wherein all functions of the apparatus are regulable directly by control means on the apparatus.

9. The apparatus according to claim 1, wherein all function are regulable remotely, preferably by radio transmission.

10. (canceled)

11. The apparatus according to claim 5, wherein the evaporative surface is a wick

12. A method of disseminating into an atmosphere at least one volatile liquid selected from at least three liquids provided in a dissemination device, comprising the steps of
providing an air current generated by a source of air current through a plurality of coplanar air channels the plurality of air channels correspond in number to the number of volatile liquids,
providing individual channels extending radially from the source of air current to one of a plurality of volatile liquid-emitting members and to the atmosphere,
blocking selectively any channel between the air current flow and the any volatile liquid-emitting member.
